(19) United States
(12) Patent Application Publication
(10) Pub. No.: US 2018/0318325 A1
(43) Pub. Date: Nov. 8, 2018

(54) ANTIBODIES FOR TARGETING CANCER STEM CELLS AND TREATING AGGRESSIVE CANCERS

(71) Applicant: GlycoMimetics, Inc., Rockville, MD (US)

(72) Inventor: John L. MAGNANI, Gaithersburg, MD (US)

(73) Assignee: GlycoMimetics, Inc., Rockville, MD (US)

(21) Appl. No.: 15/773,055

(22) PCT Filed: Nov. 2, 2016
(86) PCT No.: PCT/US2016/060088

§ 371 Date: May 2, 2018

Related U.S. Application Data
(60) Provisional application No. 62/250,406, filed on Nov. 3, 2015.

(51) Int. Cl. A61K 31/7034; G01N 33/574; A61K 45/06; A61K 38/05

(52) U.S. Cl. CPC A61K 31/7034; G01N 33/57492; A61K 45/06; G01N 2333/70564; G01N 33/57426; G01N 33/57407; A61K 38/05

(57) ABSTRACT

Methods and systems for identifying and treating patients with cancers that can bind E-selectin are disclosed. E-selectin-binding cancers are identified by their cell surface expression sialyl Le³ and sialyl Le³ carbohydrate epitopes, and such cancers can be identified by antibodies that bind to sialyl Le^{a/x}, such as HECA-452. Such cancers can be treated with antagonists of E-selectin such as glycomimetic compounds and with immunotherapies targeting the cell surface carbohydrates containing the sialyl Le^{a/x} domains to block and/or disrupt the binding of E-selectin.

This application claims the benefit under 35 U.S.C. § 119(e) to U.S. Provisional Application No. 62/250,406 filed Nov. 3, 2015, which application is incorporated by reference herein in its entirety.

The present disclosure provides methods and systems for treating patients with aggressive cancers, including drug-resistant cancers, cancers with a high likelihood of relapse, cancers with accelerated disease progression, and/or cancers with reduced survival. The disclosure also provides methods and compositions for identifying cancer stem cells and/or aggressive cancer cells (e.g., cancer cells likely to be drug resistant, cancers with a high likelihood of causing a patient to relapse, cancers likely to result in an accelerated disease progression, and/or cancers associated with reduced survival), and for the treatments of such cancers by blocking and/or disrupting certain cell surface carbohydrates (cell surface binding sites). Methods and compositions for identifying such cancers using blood samples, including blood fraction samples (e.g., plasma or serum samples), are also disclosed.

Not all cancer cells are alike. Even within a group of related cancer cells (e.g., a multiple myeloma cell line or prostate cancer tumor), gene expression and cell surface epitopes vary. Certain cancer cells, referred to as cancer stem cells, can establish new tumors, and the presence of higher numbers of these stem cells in a patient are associated with poorer prognoses. These cancer stem cells may also exhibit the more aggressive cancer traits such as drug resistance, accelerated disease progression, shorter survival, and higher incidence of relapse. Identifying cancer stem cells and eliminating these cells from patients has been a challenge. The following may provide a means to overcome this challenge.

Cancer stem cells have been found to express cell surface carbohydrates that can bind to E-selectin. The cell surface carbohydrates that can bind E-selectin contain carbohydrate epitopes known as sialyl Le^{a} and sialyl Le^{x} carbohydrates. These sialyl Le^{a} and sialyl Le^{x} carbohydrates have also been found to bind to the monoclonal antibody HECA-452. That is, there is a trisaccharide domain common to both sialyl Le^{a} and sialyl Le^{x} (sialyl Le^{x}) that binds to both E-selectin and the HECA-452 antibody. See Berg et al., “A Carbohydrate Domain Common to Both Sialyl Le^{a} and Sialyl Le^{x} Is Recognized by the Endothelial Cell Leukocyte Adhesion Molecule ELAM 1,” J. Biol. Chem. (1991) 266:14869-72, which is hereby incorporated by reference. Cancer cells that can bind to E-selectin are capable of resisting certain standard treatments for cancer, such as chemotherapy. That is, cancer cell populations that can bind to the sialyl Le^{a/x} domain (and thus, can also bind to E-selectin) are correlated with drug-resistance, accelerated disease progression, shorter survival, and higher incidence of relapse. It is thought that cancer cells expressing the carbohydrate epitope that binds antibodies with the sialyl Le^{a/x} binding domain (e.g., cancer cells that can bind the HECA-452 antibody) are able to survive chemotherapy treatment because they are also able to bind to E-selectin expressed on the vascular endothelium. Thus, for example, when bound to the E-selectin in the protective niches of bone marrow, these cancer cells are able to survive cancer treatments such as chemotherapy. These cancer cells may be detected directly (e.g., binding to the cancer cells themselves), or indirectly (e.g., detecting molecules in blood associated with these cancers).

In accordance with this disclosure, methods and compositions are provided for the discovery and production of antibodies that bind to sialyl Le^{a/x} that can be used to identify cancer stem cells. A number of cancer treatments utilizing these methods and compositions are also provided herein. In particular, the antibodies provided in the instant disclosure are able to identify cancer cell populations expressing the cell surface carbohydrates that also bind E-selectin. This identification may be direct (e.g., detection of the cell expressing the cell surface carbohydrate) or indirect (e.g., detection of the carbohydrate epitope on molecules secreted or otherwise present in the blood). Any antibody, oligonucleotide or peptide molecule, for example an aptamer or affimer, that binds sialyl Le^{a/x} could be used to identify cancer cell populations expressing the cells surface carbohydrate that also binds E-selectin (or to identify the carbohydrate epitope present in blood).

Based on the binding of the disclosed sialyl Le^{a/x}-binding antibody either to the cancer cells or to the carbohydrate epitope on molecules present in the blood, cancer patients with aggressive cancers can be identified. The instant disclosure contemplates treating patients with cancers that express the cell surface carbohydrate or that produce the carbohydrate epitope on molecules in the blood with the epitope common to sialyl Le^{a} and sialyl Le^{x} with therapies that interfere with the function of that cell surface carbohydrate. In particular, it is thought that by blocking or otherwise inhibiting E-selectin, E-selectin is unable to bind to the tumor cell surface carbohydrate. Without being able to bind to E-selectin, the cancer stem cells are unable to become chemoresistant or to hide in protective niches in bone marrow and unable escape chemotherapy treatment. Examples include treatment with compounds, such as glycomimetic compounds, or immunotherapies that target the cell surface carbohydrates that bind to antibodies with the epitope common to sialyl Le^{a} and sialyl Le^{x} and to interfere with that cell surface carbohydrate's functions. Glycomimetic compounds suitable for such treatments may comprise the following Formula (I):

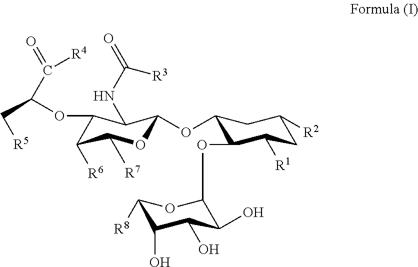

wherein R¹ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups; R² is chosen from H, a non-glycomimetic moiety, and a linker-non-glycomimetic moiety, wherein the non-glycomimetic moiety is chosen from polyethylene glycol, N-linked cyclam, thiazolyl, chromenyl, —C(═O)NH(CH₂)₁₋₄NH₂, C₁-C₈ alkyl, and —C(═O)OY groups, wherein Y is chosen from C₁-C₄ alkyl, C₂-C₄ alkenyl, and C₂-C₄ alkynyl groups; R³ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups; R⁴ is chosen from —OH and —NZ¹Z² groups, wherein Z¹ and Z², which may be identical or different, are each independently chosen from H, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups, wherein Z¹ and Z² may join together to form a ring; R⁵ is chosen from C₃-C₈ cycloalkyl groups; R⁶ is chosen from —OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups; R⁷ is chosen from —CH₂OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups; and R⁸ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups.

Suitable compounds for such treatment may also include prodrugs of Formula (I) and pharmaceutically acceptable salts of any of the foregoing. The present disclosure includes within its scope all possible tautomers. Furthermore, the present disclosure includes in its scope both the individual tautomers and any mixtures thereof.

In some embodiments, R¹, R³, R⁴, R⁵, R⁶, R⁷, and R⁸ are as defined above, and R² is a linker-non-glycomimetic moiety, wherein the non-glycomimetic moiety comprises polyethylene glycol. Glycomimetic E-selectin antagonists, such as, for example, the glycomimetic E-selectin antagonists disclosed in U.S. Pat. No. 9,109,002, which is hereby incorporated by reference, may be suitable for use in such treatment. One such glycomimetic compound is GMI-1271.

In some embodiments, R¹, R³, R⁴, R⁵, R⁶, R⁷, and R⁸ are as defined above, and R² is a linker-non-glycomimetic moiety, wherein the non-glycomimetic moiety comprises a N-linked cyclam. Glycomimetic heterobifunctional compounds that are antagonists of E-selectin and CXCR4 such as, for example, those disclosed in U.S. Pat. No. 8,410,066, which is hereby incorporated by reference, may be suitable for use in such treatment. One such glycomimetic compound is GMI-1359. See, e.g., Steele, Maria M. et al., “A small molecule glycomimetic antagonist of E-selectin and CXCR4 (GMI-1359) prevents pancreatic tumor metastasis and improves chemotherapy [abstract],” Proceedings of the 106th Annual Meeting of the American Association for Cancer Research, 2015 Apr. 18-22, Philadelphia, Pa.; Philadelphia (Pa.): AACR, Cancer Res 2015, 75(15 Suppl):Abstract nr 425. doi:10.1158/1538-7445.AM2015-425; Gravina, Giovanni L. et al., “Dual E-selectin and CXCR4 inhibition reduces tumor growth and increases the sensitivity to docetaxel in experimental bone metastases of prostate cancer [abstract],” Proceedings of the 106th Annual Meeting of the American Association for Cancer Research, 2015 Apr. 18-22, Philadelphia, Pa.; Philadelphia (Pa.): AACR, Cancer Res 2015, 75(15 Suppl):Abstract nr 428. doi:10.1158/1538-7445.AM2015-428, all of which are incorporated by reference.

### BRIEF DESCRIPTION OF THE DRAWINGS

FIG. 1A is a graph of a population of MM1S^{parental} cells, with the CD138 marker for myeloma cells of the MM1 S^{parental} cells indicated on the Y-axis and the MM1S^{parental} cells positive for HECA-452 indicated on the X-axis.

FIG. 1B is a graph of a population of MM1S^{HECA452} cells, with the CD138 marker for myeloma cells of the MM1S^{HECA452} cells indicated on the Y-axis by and the MM1S^{HECA452} cells positive for HECA-452 indicated on the X-axis.

FIG. 2 is a graph of survival rates of female SCID mice injected with MM1S^{parental} cells and female SCID mice injected with MM1S^{HECA452} cells.

FIG. 3 is a graph of survival proportions of mice injected with MM1S^{parental} cells and treated with GMI-1271, bortezomib (BTZ), and GMI-1271 and bortezomib, with saline as a control.

FIG. 4 is a graph of survival proportions of mice injected with MMIS^{HECA452} cells and treated with GMI-1271, bortezomib, and GMI-1271 and bortezomib, with saline as a control.

FIG. 5 is a graph of the number of human CD138+ MM cells mobilized into the bloodstream in mice engrafted with MM1S^{HECA452} over time after 1 dose of GMI-1271.

FIG. 6 is a graph of the expression of E-selectin ligands detected by mAb HECA-452 by AML blasts obtained from newly diagnosed patients compared with AML blasts of relapsing patients.

FIG. 7 provides a conceptual representation of the HECA-452 capture/CD-B assay for detecting cancer markers, including markers for cancer stem cells and/or aggressive cancer cells, in blood serum.

FIG. 8 provides a conceptual representation of the CD-B capture/HECA-452 assay for detecting cancer markers, including markers for cancer stem cells and/or aggressive cancer cells, in blood serum.

FIG. 9 shows the percentage of KG1 and KG1a cells that express HECA-452 (i.e., that are HECA-452 positive), as detected by flow cytometry.

FIG. 10A shows the amount of ligands in KG1 conditioned media that bind to both HECA-452 antibodies and CD62L antibodies using the HECA-452/CD62L sandwich ELISA assay.

FIG. 10B shows the amount of ligands in KG1 conditioned media that bind to HECA-452 antibodies using the HECA-452/HECA-452 sandwich ELISA assay.

FIG. 11 the amount of various ligands in KG1a conditioned media that bind to both HECA-452 antibodies and the various detection antibodies ((CD33, CD62L, CD123, CD43, CD44, and CD147 detection antibodies) using a HECA-452/detection antibody sandwich ELISA assay.

FIG. 12A the amount of ligands in KG1 conditioned media and the amount of ligands in KG1a conditioned media that bind to both CD62L antibodies and HECA-452 antibodies using a CDL62L capture/HECA-452 detection ELISA assay.

FIG. 12B shows the results (absorbance at 450 nM) indicating the amount of ligands in KG1 conditioned media and the amount of ligands in KG1a conditioned media that bind to both HECA-452 antibodies and CD62L antibodies using a HECA-452 capture/CD62L detection ELISA assay.

Reference will now be made in detail to the present embodiments (exemplary embodiments) of the disclosure, examples of which are illustrated in the accompanying drawings. Wherever possible, the same reference numbers will be used throughout the drawings to refer to the same or like parts.

The abbreviations used herein generally have their conventional meaning in the chemical and biological arts.

The term “antibody,” “antibodies,” “ab,” or “immunoglobulin” are used interchangeably in the broadest sense and include monoclonal antibodies, including isolated, engineered, chemically synthesized or recombinant antibodies (e.g., full length or intact monoclonal antibodies), and also antibody fragments, oligonucleotides or peptide molecules (e.g., aptamers or affimers) so long as they exhibit the desired biological activity. In one embodiment, the disclosure relates to monoclonal antibodies.

An antibody molecule consists of a glycoprotein comprising at least two heavy (H) chains and two light (L) chains inter-connected by disulfide bonds. Each heavy chain comprises a heavy chain variable region (or domain) (abbreviated herein as HCVR or VH) and a heavy chain constant region. The heavy chain constant region comprises three or four domains, CH1, CH2, CH3, and CH4. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order; FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g. effector cells) and the first component (Clq) of the classical complement system.

By “antigen binding fragment” of an antibody according to the disclosure, it is intended to indicate any peptide, polypeptide, or protein retaining the ability to bind to the target of the antibody. In one embodiment, the target is selected from sialyl Le^{a}, sialyl Le^{x}, sialyl Le^{a/x}, and/or an E-selectin ligand. In certain embodiments, antigen binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies.

The term “monoclonal antibody” or “Mab” as used herein refers to an antibody obtained from a population of substantially homogeneous antibodies, i.e. the individual antibodies of the population are identical except for possible naturally occurring mutations that may be present in minor amounts. Typically, monoclonal antibodies are highly specific, being directed against a single epitope. Such a monoclonal antibody can be produced by a single clone of B cells or hybridoma, Monoclonal antibodies can also be recombinant, i.e., produced by protein engineering. Monoclonal antibodies can also be isolated from phage antibody libraries. In addition, in contrast with preparations of polyclonal antibodies which typically include various antibodies directed against various determinants, or epitopes, each monoclonal antibody is directed against a single epitope of the antigen. The disclosure relates to an antibody isolated or obtained by purification from cells or obtained by genetic recombination or chemical synthesis.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, such as, for example, a polypeptide determinant or a carbohydrate determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. In one embodiment, an antibody is said to specifically bind an antigen when the dissociation constant is less than or equal to about 1 μM, such as, for example, when the dissociation constant is less than or equal to about 100 nM, such as, for example, when the dissociation constant is less than or equal to about 1 nM, and such as, further for example, when the dissociation constant is less than or equal to about 100 pM. The terms “specific for” and “specific binding,” as used herein, are interchangeable and refer to antibody binding to a predetermined antigen, e.g., the epitope common to sialyl Le^{a}, sialyl Le^{x}, and sialyl Le^{a/x}. Typically, the antibody binds with a dissociation constant (K_{D}) of 10⁻⁶ M or less, and binds to the predetermined antigen with a K_{D} that is at least twofold less than its K_{D} for binding to a nonspecific antigen (e.g., BSA, casein, or any other specified polypeptide) other than the predetermined antigen. The phrases “an antibody recognizing an antigen” and “an antibody specific for an antigen” are used interchangeably herein with the term “an antibody which binds specifically to an antigen.”

As used herein, “expansion” includes any increase in cell number. Expansion includes, for example, an increase in the number of hematopoietic stem cells over the number of HSCs present in the cell population used to initiate the culture.

Treatment with drugs that interfere with the binding of E-selectin to the sialyl Le^{a} or sialyl Le^{x} epitope may be used to improve the efficacy of other cancer treatments, such as chemotherapy. In particular, liquid cancers, such as multiple myeloma, and solid cancers, such as prostate cancer, are candidates for such identification of aggressive subpopulations of sialyl Le^{a}, and sialyl Le^{x}-binding cancer cells and treatment with drugs that interfere with the cell surface carbohydrates containing sialyl Le^{a} and sialyl Le^{x} epitopes, thereby interfering with the cell's binding to E-selectin.

Multiple Myelomas arise from the transformation of plasma cells which are the fully differentiated cell type of the B-cell lineage. Other blood cancers arise from cells early in the differentiation of normal B-cells, such as the early and pre-B-cells. These transformed cells from the early B-cell lineage are known as acute lymphocytic leukemia (ALL). In contrast to plasma cells and multiple myelomas, pre-B cells and ALL cells are heavily glycosylated with the antigens for HECA-452 (i.e. sialyl Le^{a/x}) suggesting that this antigen is developmentally regulated in this B-cell lineage. In fact, Sipkins et al., Nature 435: 969-973 (2005), which is hereby incorporated by reference, demonstrated that the ALL cell line NALM-6 is glycosylated with this carbohydrate epitope that allows the cells to bind to E-selectin expressed in the microdomains of the bone marrow vasculature.

The developmentally regulated glycosylation by sialyl Le^{x} of the B-cell lineage and their transformed lymphomas was evaluated by Kikuchi et al., Glycobiology 15: 271-280 (2005), which is hereby incorporated by reference. Kikuchi et al. showed that sialyl Le^{x} is expressed on cells in the early developmental stages of the B-cell lineage and is lost upon differentiation. This glycosylation pattern is mirrored by the lymphomas that arise from these developmental stages. Thus, if clonogenic early stage B cells represent multiple myeloma stem cells, they should express the epitope common to sialyl Le^{a} and sialyl Le^{x}. These MM stem cells should then also functionally bind E-selectin.

As shown in examples 1-4 below, the instant disclosure confirms that certain subpopulations of MM cells express functional E-selectin ligands. That is, about 5% to about 10% of MM cells express E-selectin ligands sialyl Le^{a} and sialyl Le^{x}. The percentage of MM cells that express E-selectin ligands can be increased under hypoxic conditions similar to the hypoxic conditions in bone marrow.

Furthermore, as shown in examples 5-8 below, the instant disclosure further confirms that these E-selectin ligands are secreted in detectable levels.

### EXAMPLES

### Example 1

### E-Selectin Ligand Expression in MM Cells

The present disclosure provides information on the cell surface carbohydrate expression on multiple myeloma cell line MM1S. Cell surface carbohydrate expression was determined by binding anti-carbohydrate antibodies followed fluorescence activated cell sorter (FACS) analysis. As shown in Table 1, The majority of MM1S cells expressed the Le^{x} carbohydrate epitope (e.g., over 90%), while a much smaller subpopulation (˜5-10%) expressed the sialylated Le^{x} epitope as determined by binding antibody HECA-452, an E-selectin/hlg chimera, and other anti-carbohydrate antibodies. In this way, the MM1S cells that express E-selectin ligands were identified by their binding to the HECA-452 antibody.

TABLE 1

MM1S
Cells

| Marker | % | MFI |
|---|---|---|
| CD15 (Le^{x}) | 97 | 73 |
| CD15s (sLe^{x}) | 11 | 9.6 |
| CD18 (β2 Integrin) | 2.1 | 4.1 |
| CD29 (β1 Integrin) | 3.9 | 6.2 |
| CD34 | 14 | 11 |
| CD44 | 88 | 21 |
| CD65 | 11 | 8.0 |
| HECA-452 (sLe^{a/x}) | 2.5 | 5.3 |
| GSLA2 (Le^{a}) | 13 | 11 |
| FH6 (extended Le^{x}) | 5.7 | 5.0 |
| E-selectin-Fc Binding | 6.4 | 6.2 |
| P-selectin-Fc binding | 60 | 32 |
| Anti-L-selectin | 2.7 | 4.5 |

### Example 2

### Murine Transplant Model—Multiple Myeloma

GMI-1271, a small molecule glycomimetic antagonist to E-selectin, has previously been administered to MM cells from the MM1S cell line that bind to HECA-452. The application of GMI-1271 has blocked the rolling of the MM1S cells on E-selectin. GMI-1271 administration has also been found to enhance the activity of bortezomib (an anti-myeloma drug) in in vivo murine transplant models (see Natoni et al., Blood, 2014, which is hereby incorporated by reference).

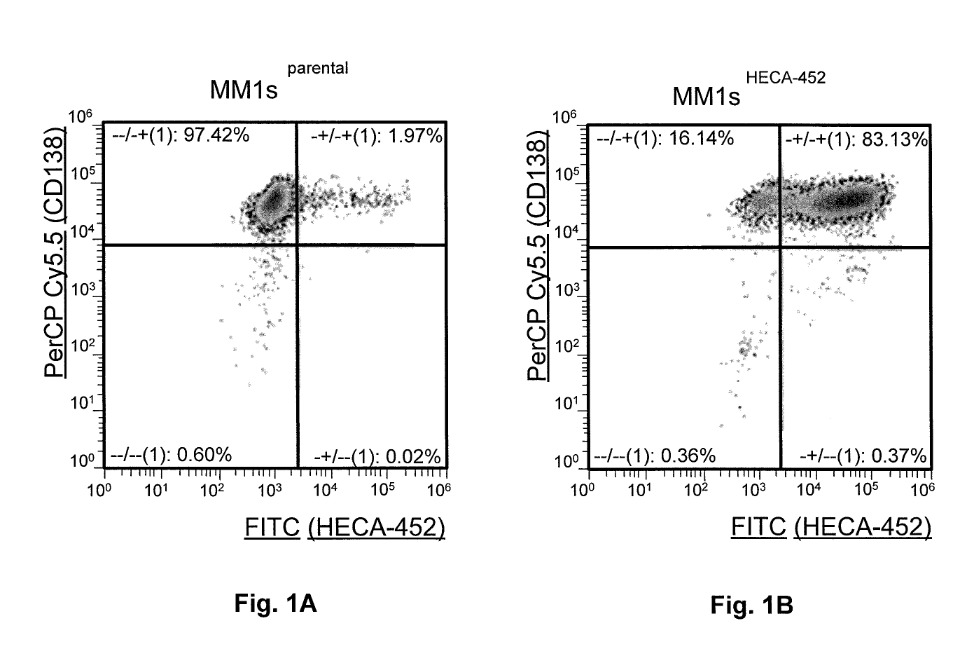

The parental, heterogeneous MM cell lines MM1S and RPM18226 (MM1S^{par}, RPMI8226^{par}, respectively) were sequentially sorted to obtain cell lines highly enriched (>85%) for the expression of cell surface carbohydrates bound by HECA-452 (MM1S^{HECA452}, RPMI8226^{HECA452,} respectively). FIGS. 1A and 1B provide the data supporting the sorting of the MM1S cells to obtain HECA-452-positive cells used to expand the MM1S^{HECA452} cell line. For example, FIG. 1A shows the parent MM1S population with approximately 5% of cells positive for HECA-452. The MM1s^{HECA-452} cells are approximately 85% positive for HECA-452, as shown in FIG. 1B. FIGS. 1A and 1B include CD138 as a marker for viable myeloma cells.

The derived cell lines could be passaged in vitro and were stable for enriched E-selectin ligand expression identified by antibody HECA-452. Both MM1S^{HECA452} and RPM18226^{HECA452} showed strong binding to E-selectin in static adhesion assays in contrast to parental cells, which showed minimal adhesion. MM1S^{HECA452} cells showed clear morphologic changes on binding to E-selectin, spreading out and becoming less reflective, in contrast to parental cells, which remained non-adherent, round and retractile. Both MM1S^{HECA452} and RPMI8226^{HECA452} exhibited strong rolling on E-selectin under shear stress, mimicking physiologic blood flow. MM1S^{par} or RPMI8226^{par} failed to roll well on E-selectin. The inclusion of GMI-1271 during culture conditions led to a marked reduction in adhesion of MM1S^{HECA452} and profoundly inhibited rolling on E-selectin of both HECA-452 enriched and parental MM cell lines.

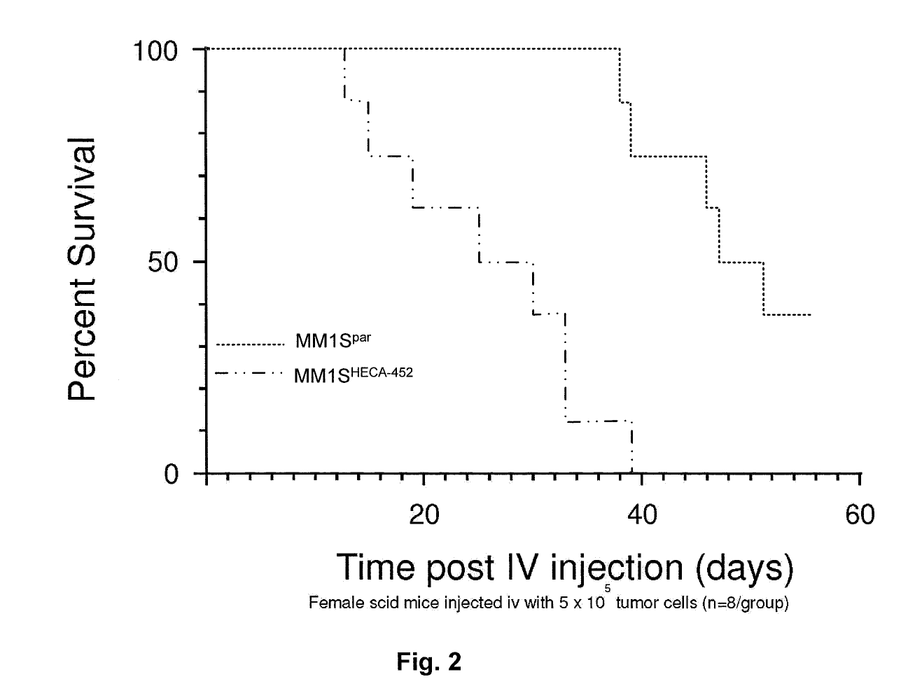

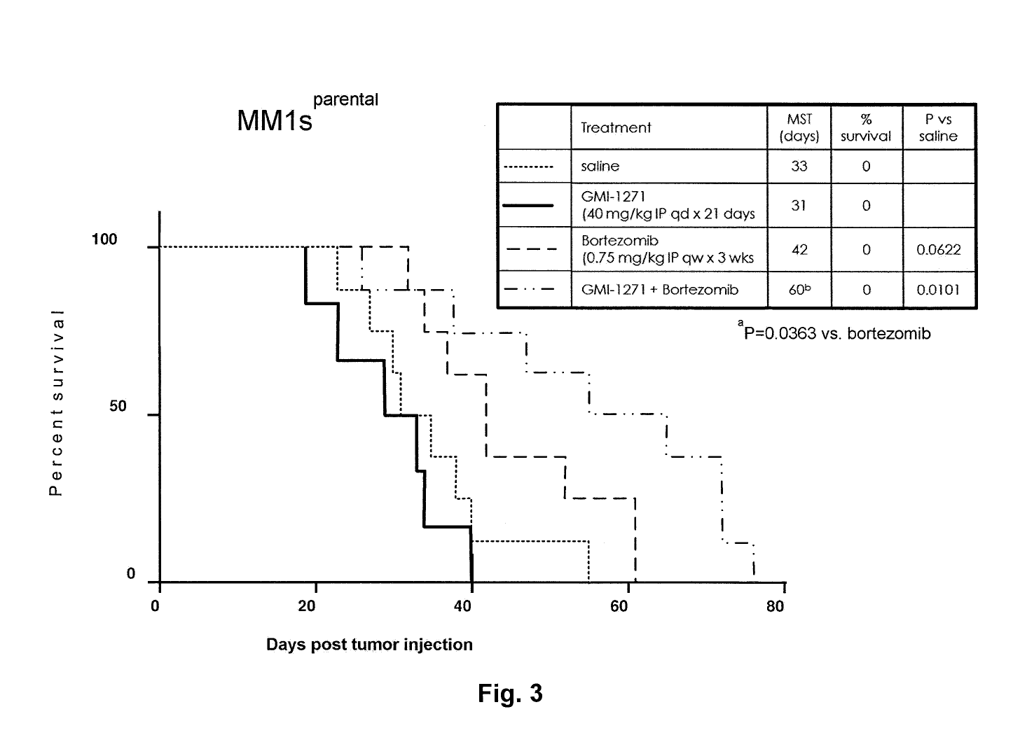

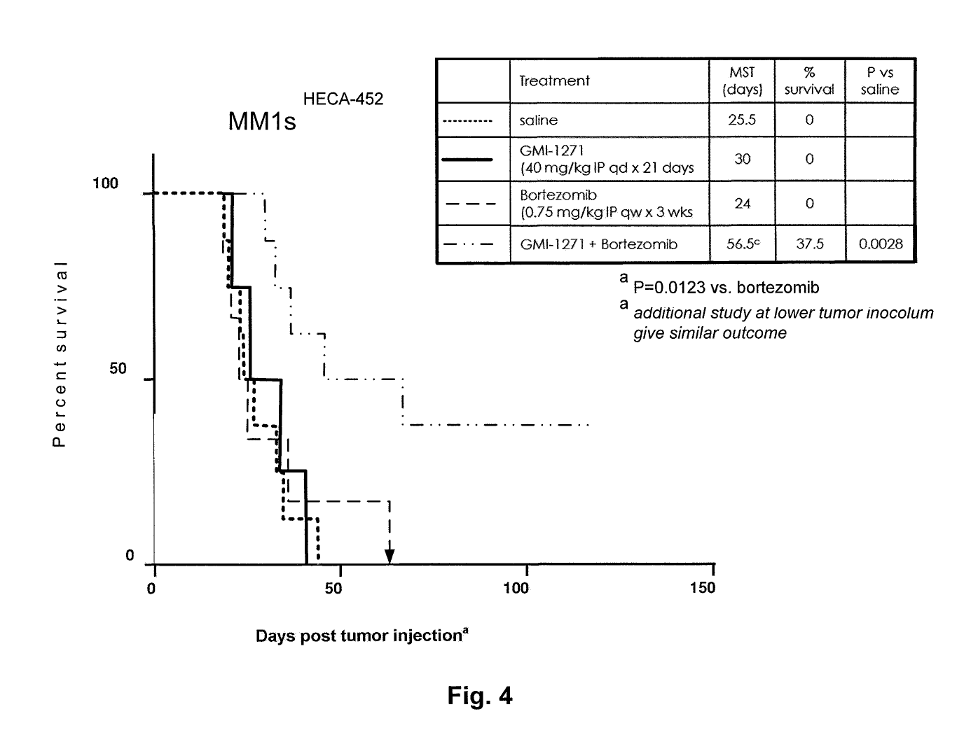

The significance of these in vitro findings were studied in vivo. Female SCID beige mice were injected i.v. with either MM1S^{par} or MM1S^{HECA452} (5×10⁵ cells, n=8/group) and followed for survival (see, e.g., FIG. 2). In separate cohorts, the effect of treatment with saline control, GMI-1271, bortezomib (BTZ) or a combination of both was determined in mice transplanted with either MM1S^{par} or MM1S^{HECA452} cells. As shown in FIG. 2, mice transplanted with MM1S^{HECA452} had more aggressive disease with significantly shorter survival compared to those transplanted with MM1S^{par}. In contrast to the parental cell line (see FIG. 3), mice engrafted with MM1S^{HECA452} demonstrated a marked resistance to BTZ treatment (see FIG. 4). As shown in FIG. 3, whereas GMI-1271 treatment alone had no impact on survival, the combination of GMI-1271 and BTZ led to a highly significant improvement in survival of MM1 S^{par} engrafted mice (P=0.0363). Importantly, as shown in FIG. 4, the combination of GMI-1271 and BTZ broke the resistance and restored the anti-myeloma activity of BTZ in MM1S^{HECA452} engrafted mice (P=0.0028).

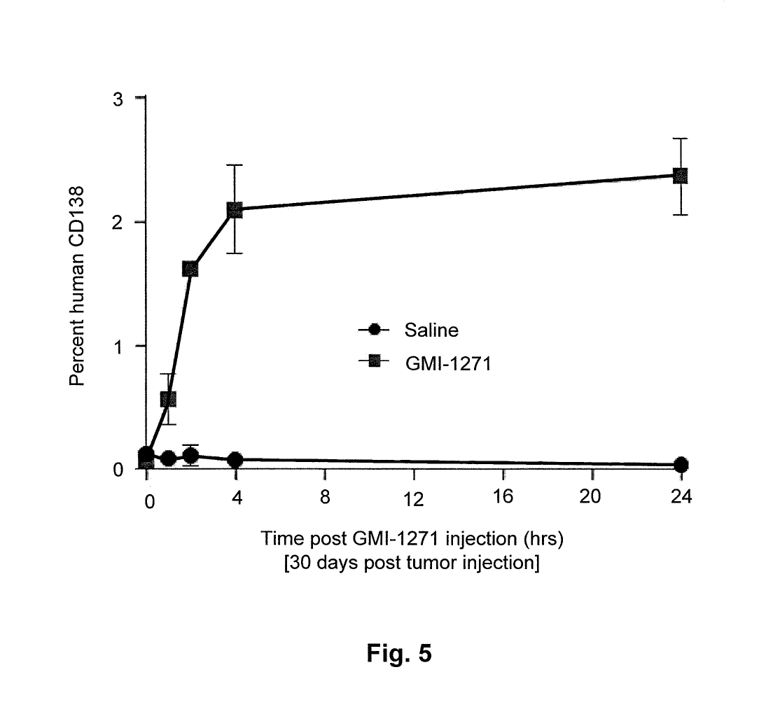

The number of human CD138+ MM cells was mobilized into the bloodstream in mice with MM1S^{HECA452} tumors within 60 min following a single injection of GMI-1271 (see, e.g., FIG. 5) and persisted for at least 24 hours (2.37% v. 0.03%, p<0.001). This effect was consistent with GMI-1271 disrupting the tumor microenvironment and mobilizing MM1S^{HECA-452} cells from the BM niche into the peripheral blood.

### Example 3

### Human Multiple Myeloma and E-Selectin Ligand Expression

Given these findings, the expression of E-selectin ligands from samples of MM cells obtained from human patients were studied, and the correlation between the levels of E-selectin expression and disease progression were determined. Bone marrow (BM) and/or peripheral blood (PB) were obtained following informed consent from patients with MM. Plasma cells (CD38+/CD138+) were analyzed for E-selectin ligand expression by flow cytometry using the HECA-452 antibody. All primary MM samples (n=25) contained HECA-452-reactive cell populations (median 22%). A consistently higher proportion of circulating MM cells isolated from patient PB express HECA-452 when compared with paired BM samples (n=14), with a median difference of 33% (Wilcoxon signed rank test, p=0.02). HECA-452 expression of MM in PB was significantly higher (on average 40% higher) in samples taken at relapse vs. diagnosis, (unpaired t test, p=0.0008)

These studies indicate that E-selectin ligand-bearing cells may play an important role in dissemination, disease progression, and/or drug resistance in cancers such as MM. Accordingly, clinical strategies incorporating glycomimetic compounds such as those disclosed in U.S. Pat. No. 9,109,002 and incorporated herein by reference may improve patient outcome.

### Example 4

### E-Selectin Ligand Expression in Acute Myelogenous Leukemia (AML) Cells

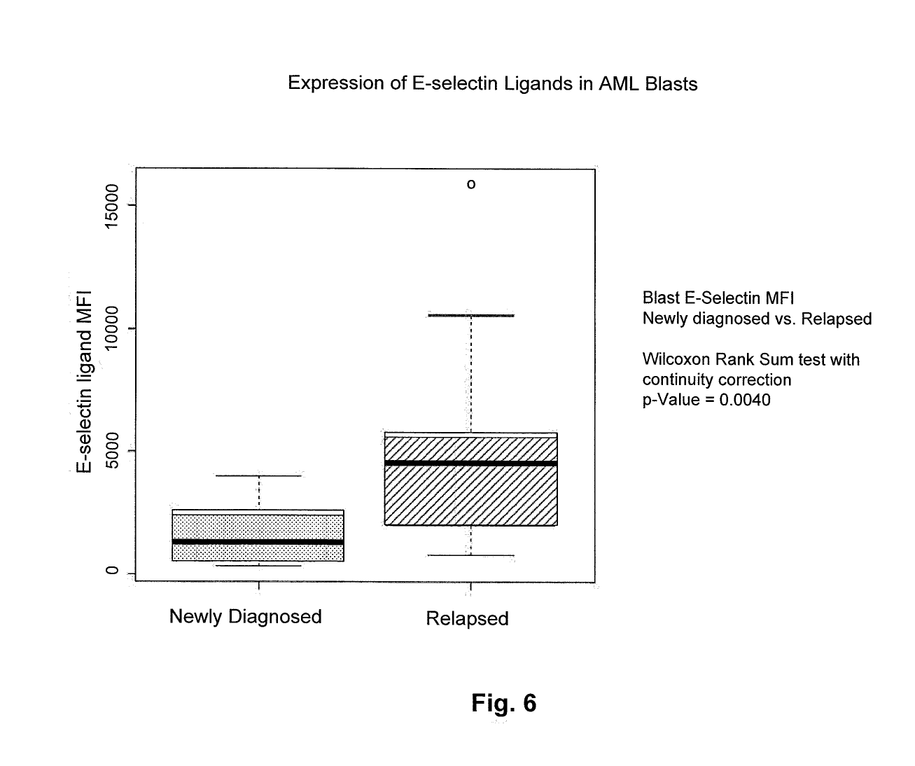

Relapse in AML patients is thought to arise from leukemic stem cells that escaped chemotherapy treatment within the protective niches in the bone marrow, presumably by binding to E-selectin. According to this mechanism, surviving relapsed cells should be selected for expression of the E-selectin ligand which is detectable by antibodies that bind sialyl Le^{a} and sialyl Le^{x}, such as the HECA-452 antibody. When AML blasts from patients were assayed for cell surface expression of the HECA-452 epitope, those cells from patients undergoing relapse of the AML cancer expressed significantly greater HECA-452 antigen on the cell surfaces than AML blasts obtained from newly diagnosed AML patients. The results of this study are provided in the graph in FIG. 6.

### Example 5

### ELISA Blood Serum Assay—Experimental Procedures

The present disclosure also provides information on the carbohydrate containing the sialyl Lex or sialyl Lea epitope expressed on cancerous cells, the secretion or release of those expressed carbohydrate epitopes on molecules into blood, including blood fractions such as plasma or serum, and the detection of the secreted carbohydrate epitopes on molecules in blood to detect cancers, including cancer stem cells and/or aggressive cancers. An overview of the ELISA Sandwich HECA-452 capture/CD-B detect assay procedure is provided below. A similar procedure was followed for the CD-B capture/HECA-452 detection assay, substituting the CD-B capture antibody for the HECA-452 capture antibody (and vice-versa).

Microplates were coated with HECA-452 capture antibody overnight with a carbonate buffer. The coating buffer was then discarded and wells were washed with ELISA wash buffer, incubated for three minutes, and then washed again. ELISA blocking buffer was then added and the microplates were then incubated one hour at ambient temperature with slow shaking. The serum test sample was then diluted with sample diluent buffer. The blocking buffer was discarded and the test samples were immediately added to the blocked wells without washing. The microplates were then incubated for two hours with slow shaking. The test sample was discarded and the wells were washed with ELISA wash buffer and incubated for 3 minutes with shaking (this step repeated 3 times).

Biotin-labeled detection antibodies were prepared by dilution in sample diluent buffer. The E-selectin ligand detection antibodies were CD43 (clone MEM-59; Novus, 0.5 μg/mL final concentration), CD44 (clone F10-44-2; Novus, 0.25 μg/mL final concentration), CD62L (Sheep pAb; R&D Systems, 0.25 μg/mL final concentration), and CD147 (clone MEM-M6/1; Thermo Fisher, 0.5 μg/mL final concentration). The AML marker detection antibodies were CD 33 (clone HIM3-4; Novus, 1 μg/mL final concentration) and CD 123 (clone 6H6; Novus, 0.5 μg/mL final concentration).

Then, for each detection antibody tested, the detection antibody solution was added to each well and incubated for 1.5-2 hours at ambient temperature with slow shaking. The detection antibody solution was discarded and the wells were washed with ELISA wash buffer, incubated for 3 minutes with shaking (this step repeated 3 times). Enzyme conjugate was diluted in sample diluent buffer and added to each well and incubated for 45 minutes at ambient temperature with slow shaking. The enzyme conjugate was discarded. Wells were washed with ELISA wash buffer and incubated 3 minutes with shaking (this step repeated 3 times). TMB substrate was added to each well and incubated 15-20 minutes at ambient temperature with slow shaking. The reaction was stopped by adding a 10% phosphoric acid solution. Then the absorbance was measured using a microplate reader.

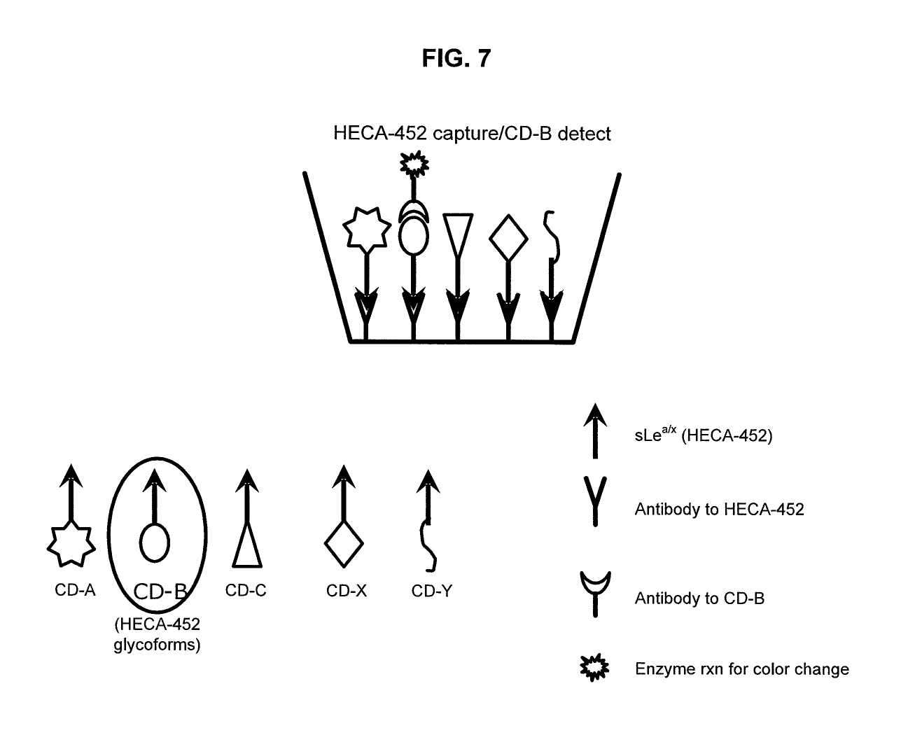

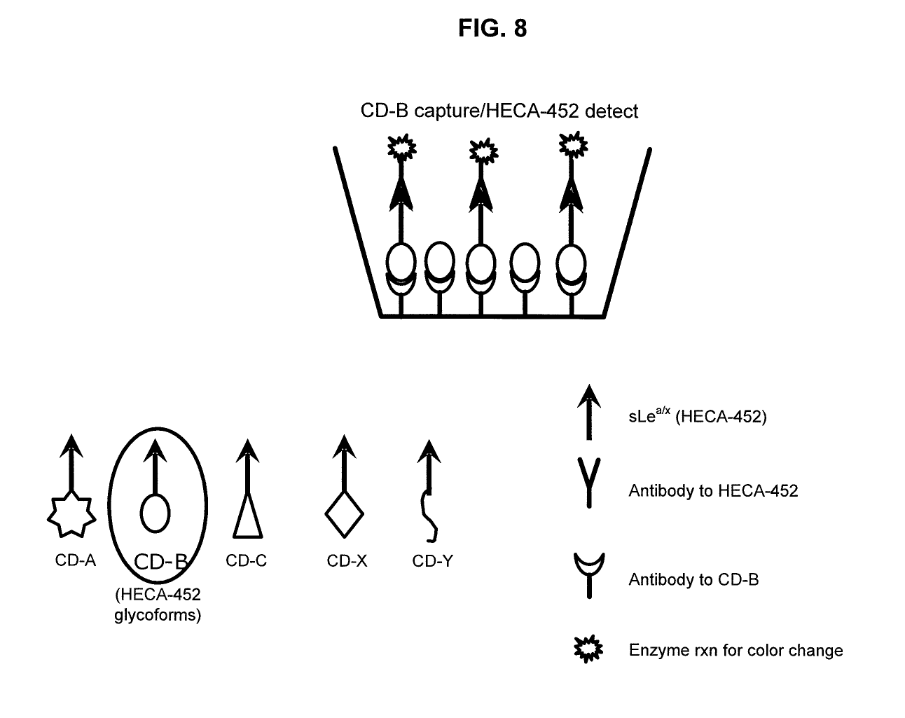

FIG. 7 provides a conceptual representation of the detect/capture assay consistent with the overview provided above, used to detect the carbohydrate epitope (using HECA-452 mAb) on the molecule (represented by CD-B and detected by an antibody to CD-B). In the assay, all serum molecules expressing the sialyl Le^{a} or sialyl Le^{x} epitopes are captured on a solid phase and the specific glycosylated molecule of interest (i.e. CD-B) is detected by an appropriate antibody (i.e. anti-CD-B). Alternatively, all molecules expressing the carbohydrate epitope sialyl Le^{a} or sialyl Le^{x} can be determined using antibody HECA-452 for both capture and detection for detecting markers of AML, including AML stem cells or aggressive AML cells, in serum. In addition, FIG. 8 provides a conceptual representation of the CD-B capture/HECA-452 detection assay for detecting markers of AML, including AML stem cells or aggressive AML cells, in serum.

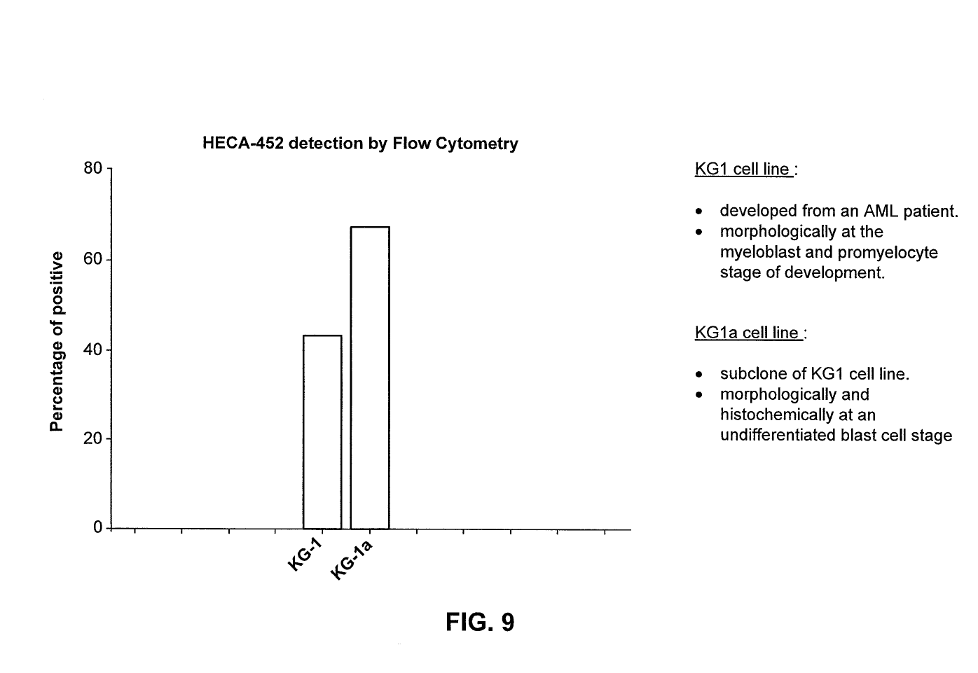

AML cell conditioned supernatant/media was also prepared for use in the experiments. In particular KG1 and KG1a cell lines were used. The KG1 cell line was developed from an AML patient and are cells that are morphologically at the myeloblast and promyelocyte stage of development. The KG1a cell line is a subclone of the KG1 cell line. It consists of cells that are morphologically and histochemically at an undifferentiated blast cell stage. Using flow cytometry, the HECA-452 expression was detected in each of these cell lines. As shown in FIG. 9, over 40% of KG1 cells are positive for HECA-452 and almost 70% of KGla cells express HECA-452. The increased glycosylation of the E-selectin carbohydrate ligand detected by antibody HECA-452 on KG1a cells is consistent with the greater cancer stem cell like properties of this subclone over the parent KG1 line.

### Example 6

### Detection of co-expression of CD62L and HECA-452 Antigen in KG1 Test Samples by Capture/Detect Sandwich ELISA Assay

The general procedure outlined for the ELISA sandwich assay in Example 5 above was used, where the HECA-452 capture antibody was used as the capture antibody and the biotin-labeled CD62L antibody or biotin-labeled HECA-452 antibody was used as the detection antibody.

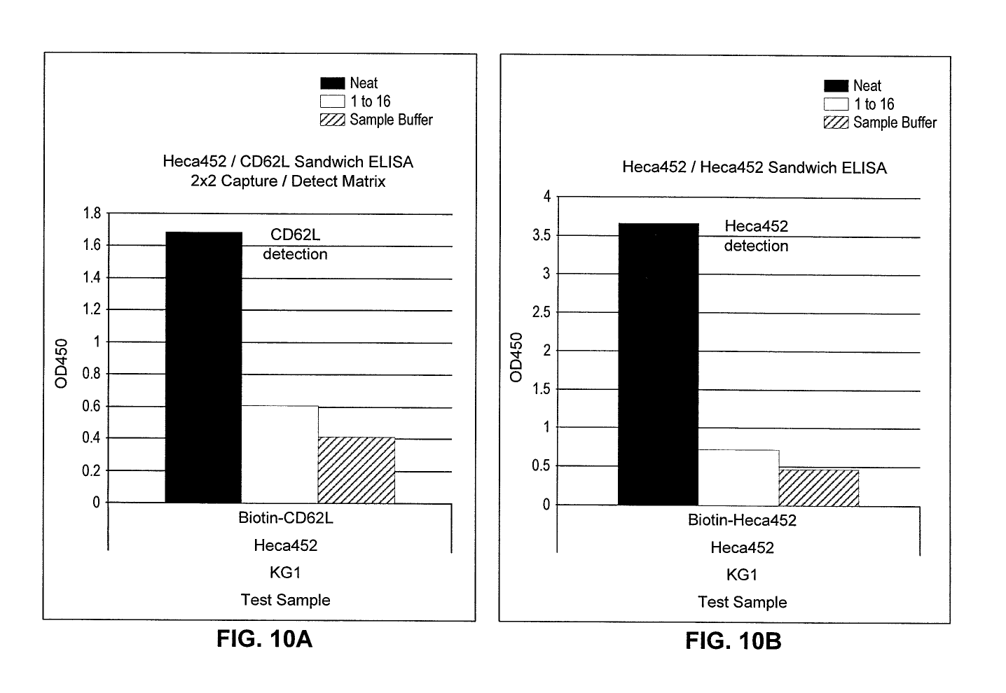

As shown in FIG. 10A, solutions of undiluted (“neat”) KG1 media, 1 to 16 dilution of KG1 media, and sample buffer were read using a microplate reader, looking at absorbance at 450 nM to determine the amount of HECA-452 glycoforms in each solution that bind to both the HECA-452 antibody and the CD62L.

As shown in FIG. 10B, solutions of undiluted (“neat”) KG1 media, 1 to 16 dilution of KG1 media, and sample buffer were read using a microplate reader, looking at absorbance at 450 nM to determine the amount of HECA-452 glycoforms in each solution that bind to both HECA-452 and CD62L.

The results show greater specificity and sensitivity detecting HECA-452 captured molecules with antibodies to CD62L (FIG. 10A) rather than with HECA-452 antibody (FIG. 10B).

### Example 7

### Detection of Various HECA-452 Glycoforms of Markers in KG1a Test Samples Using HECA-452 Capture in the ELISA Assay

The general procedure outlined for the ELISA sandwich assay in Example 5 above was used, where the HECA-452 capture antibody was used as the capture antibody and the biotin-labeled antibodies for various markers listed on the X-axis were used as the detection antibodies.

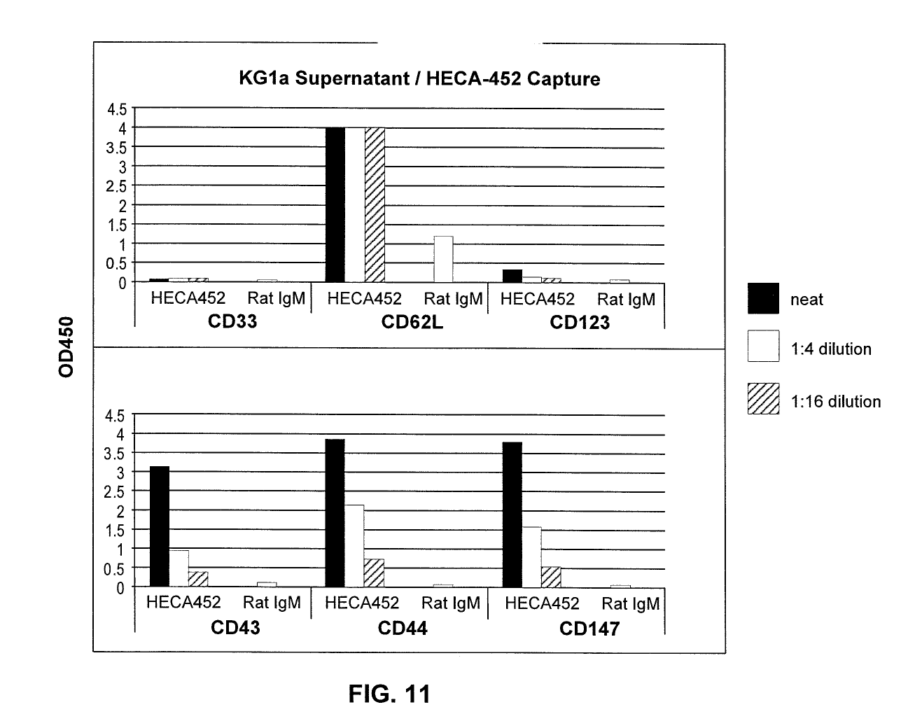

As shown in FIG. 11, solutions of undiluted (“neat”) KG1a media, 1 to 16 dilution of KG1a media, and sample buffer were read using a microplate reader, looking at absorbance at 450 nM to determine the amount HECA-452 glycoforms in each solution that bind to both HECA-452 and the respective ligand antibodies. Capture with Rat IgM was used as a negative control.

The results demonstrate that the greatest sensitivity was obtained by detecting HECA-452 glycoforms of CD62L by capturing antigens with HECA-452 mAb and detecting with antibodies to CD62L, In addition, HECA-452 glycoforms of AML markers CD33 and CD123 are not detected in the supernatant.

### Example 8

### Comparison of HECA-452 Glycoforms of CD62L in Dilutions of KG1 and KG1a Supernatants

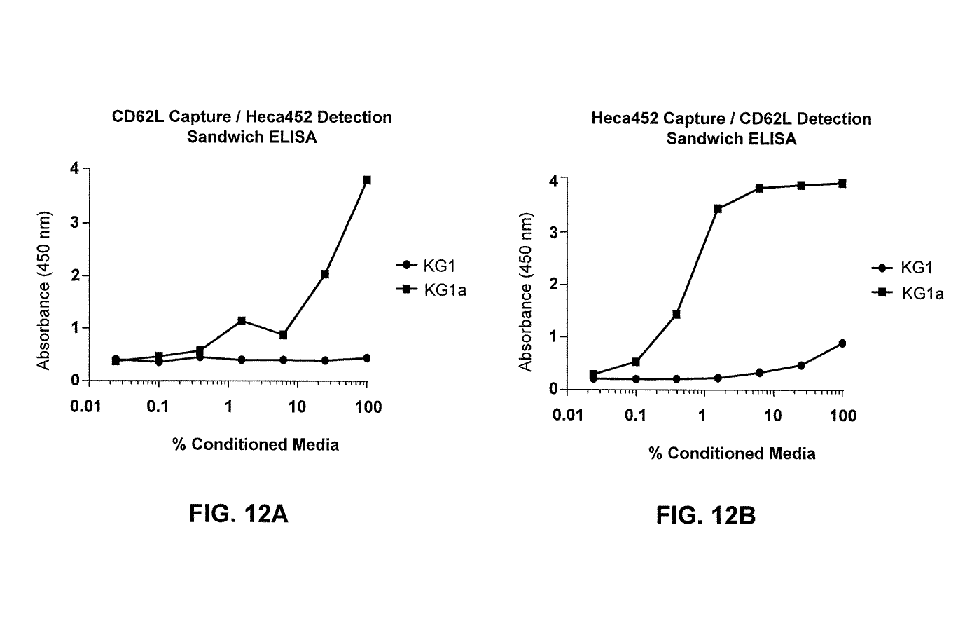

The general procedure outlined for the ELISA sandwich assay in Example 5 above was used, where the CD62L capture antibody was used as the capture antibody and the biotin-labeled HECA-452 antibody was used as the detect antibod. As shown in FIG. 12A, various dilutions of KG1 and KG1a media were read using a microplate reader, looking at absorbance at 450 nM to determine the amount of HECA-452 glycoforms in each solution that bind to both the HECA-452 antibody and the CD62L. The results show greater amounts of HECA-452 glycoforms of CD62L found in supernatants of KG1a cells in comparison to KG1 cells, which is consistent with the increased HECA-452 glycosylation (E-selectin ligands) on the surface of KG1a cells in comparison to KG1 cells as presented in FIG. 9.

For the results shown in FIG. 12A, the general procedure outlined for the ELISA sandwich assay in Example 5 above was used, where the HECA-452 capture antibody was used as the capture antibody and the biotin-labeled CD62L antibody was used as the detect antibody. As shown in FIG. 12B, various dilutions of KG1 and KG1a media were read using a microplate reader, looking at absorbance at 450 nM to determine the amount of HECA-452 glycoforms in each solution that bind to both the HECA-452 antibody and the CD62L. The results show that the format of capturing antigen with antibody HECA-452 and detecting with antibody to CD62L shows greater sensitivity than capturing antigen with antibody to CD62L and detecting with antibody to HECA-452.

### E-Selectin Ligand Expression in Solid Tumors

S. Yasmin-Karin et al., Oncotarget Oct. 6, 2014, which is hereby incorporated by reference, separated prostate cancer cells based on their ability to bind E-selectin in vitro under flow conditions. Based on their ability to bind E-selectin, these cells would also express the HECA-452 antigen on their cell surfaces. These prostate cancer cells selected for binding E-selectin displayed properties of tumor cell sternness compared with those prostate cancer cells that did not bind E-selectin. These properties included (1) colony formation in soft agar; (2) formation of tumor spheroids in vitro; (3) invasiveness into Matrigel; and (4) metastatic behavior and tumor growth and aggressiveness in vivo. These prostate cancer tumor cells, as well as other solid tumor cells that express E-selectin ligands are also candidates for identification by antibodies that bind sialyi Le^{a} and sialyl Le^{x} and treatment with glycomimetic compounds such as GMI-1271 to improve patient outcomes.

As indicated in Examples 1-4 above, direct detection of the cancerous cells of solid tumors can be accomplished using methods and compositions disclosed herein. And as indicated in Examples 5-9 above, indirect detection of such cancers by detecting secreted glycoforms in blood can also be accomplished using methods and compositions disclosed herein.

### Antibodies

The present disclosure relates to methods and compositions for the discovery and production of antibodies that can be used to identify cancer stem cells and/or aggressive cancer cells, either by directly detecting the cell-surface carbohydrates on the cells or by detecting such glycoforms secreted or otherwise present in blood.

In one embodiment, the technologies disclosed herein provide new strategies for the rapid development of diagnostic and therapeutic antibodies for the detection of aggressive cancers. In one embodiment, the technologies disclosed herein provide new strategies for the treatment of the detected aggressive cancers with compounds that interfere with the cell surface carbohydrates of the cancer cells that bind E-selectin. In one embodiment, the detected cancer cells are from a liquid cancer. In one embodiment, the detected cancer cells are from a solid tumor. In one embodiment, the cancer is detected by detecting carbohydrates present in blood. In one embodiment, the detected cancer is MM, ALL, AML, or prostate cancer. In one embodiment, the cancers are treated, after detection by the diagnostic antibodies, with a glycomimetic compound.

In one embodiment, the present disclosure relates to antibodies that can be used to identify cancer cells that express E-selectin ligands, In one embodiment, the present disclosure relates to antibodies that can be used to identify cancer ligands present in the blood. In one embodiment, the antibodies are specific for both sialyl Le^{a} and sialyl Le^{x}. In one embodiment, the antibodies detect HECA-452 glycoforms of CD62L.

In one embodiment, a population of cells is provided for the discovery and production of antibodies that can be used to identify aggressive cancer cells.

In one embodiment, the present disclosure provides antibodies that are specific for both sialyl Le^{a} and sialyl Le^{x}, the antibodies being produced by a method comprising injecting a host with cancer cells and screening the resultant antibodies for those that bind to sialyl Le^{a} and sialyl Le^{x} coated on multiwell plates. In one embodiment, the disclosure provides antibodies that are specific for both sialyl Le^{a} and sialyl Le^{x}, the antibodies being produced by a method comprising injecting a host with aggressive cancer cells (e.g., cancer cells from a relapsing patient, cancer cells from a patient that is not responding to chemotherapy, or otherwise identified aggressive cancer cells) and screening the resultant antibodies for those that bind to sialyl Le^{a} and sialyl Le^{x} coated on multiwell plates.

In one embodiment, the present disclosure provides antibodies that are specific for HECA-452 and antibodies that are specific for CD62L, which can be used together to detect HECA-452 glycoforms of CD62L, for example, as described in the assay methods described herein. The HECA-452 antibodies may be produced by a method comprising injecting mice with aggressive cancer cells (e.g., cancer cells from a relapsing patient, cancer cells from a patient that is not responding to chemotherapy, or otherwise identified aggressive cancer cells) and screening the resultant antibodies for those that bind to HECA-452 coated on multiwell plates. The CD62L antibodies may be produced by a method comprising injecting mice with aggressive cancer cells (e.g., cancer cells from a relapsing patient, cancer cells from a patient that is not responding to chemotherapy, or otherwise identified aggressive cancer cells) and screening the resultant antibodies for those that bind to CD62L coated on multiwell plates.

In one embodiment, the present disclosure provides antibodies that are specific for both HECA-452 and CD62L, and are able to detect HECA-452 glycoforms of CD62L, the antibodies being produced by a method comprising injecting mice with cancer cells and screening the resultant antibodies for those that bind to HECA-452 and CD62L coated on multiwell plates. In one embodiment, the present disclosure provides antibodies that are specific for both HECA-452 and CD62L, the antibodies being produced by a method comprising injecting mice with aggressive cancer cells (e.g., cancer cells from a relapsing patient, cancer cells from a patient that is not responding to chemotherapy, or otherwise identified aggressive cancer cells) and screening the resultant antibodies for those that bind to both HECA-452 and CD62L coated on multiwell plates.

The monoclonal antibodies (MAbs) of the disclosure can be produced by a variety of techniques, including conventional monoclonal antibody methodology, e.g., the standard somatic cell hybridization technique of Kohler and Milstein, 1975, Nature 256:495, which is hereby incorporated by reference. Somatic cell hybridization procedures may be used or other techniques for producing monoclonal antibodies can be employed, including, e.g., viral or oncogenic transformation of B-lymphocytes.

One skilled in the art can engineer mouse strains deficient in mouse antibody production with large fragments of the human Ig loci so that such mice produce human antibodies in the absence of mouse antibodies. Large human Ig fragments may preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains yields high affinity antibodies against any antigen of interest, including human antigens. Using the hybridoma technology, antigen-specific human MAbs with the desired specificity may be produced and selected.

In one embodiment, antibodies of the disclosure can be expressed in cell lines other than hybridoma cell lines. In one embodiment, sequences encoding particular antibodies can be used for transformation of a suitable mammalian host cell. In one embodiment, transformation can be achieved using any known method for introducing polynucleotides into a host cell, including, for example packaging the polynucleotide in a virus (or into a viral vector) and transducing a host cell with the virus (or vector) or by transfection procedures known in the art. Such procedures are exemplified by U.S. Pat. Nos. 4,399,216, 4,912,040, 4,740,461, and 4,959,455, which are hereby incorporated by reference. Generally, the transformation procedure used may depend upon the host to be transformed. Methods for introducing heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

In one embodiment, the disclosure provides for antibodies capable of binding to E-selectin. In one embodiment, the antibodies are HECA-452 antibodies.

In one embodiment, the disclosure provides for antibodies capable of binding specifically to E-selectin ligands expressed, or present, on a cancer cell. In one embodiment, the disclosure provides for antibodies capable of binding specifically to E-selectin ligands expressed, or present, in blood.

Screening for hybridomas/antibodies that are capable of binding specifically to sialyl Le^{a} and sialyl Le^{x} and/or specifically to E-selectin ligands (e.g., E-selectin ligands expressed by a cancer cell) can be achieved by any of a plurality of techniques available to one of ordinary skill in the art.

### Diagnostics

In one embodiment, the present antibodies also may be utilized to detect aggressive cancers and/or aggressive cancer cells in vivo or ex vivo. In one embodiment, cancer cells can be obtained from patients and analyzed ex vivo by binding cells with fluorescently labeled antibodies and analyzed by fluorescence-activated cell sorting. In one embodiment, blood can be obtained from patients and analyzed ex vivo by binding ligands with fluorescently labeled antibodies and analyzed by an ELISA assay. In one embodiment, the antibodies bind to HECA-452 and CD62L, allowing them to detect HECA-452 glycoforms of CD62L. In some embodiments, multiple antibodies are used in the detection.

Detection in vivo is achieved by labeling the antibodies described herein, administering the labeled antibody to a subject, and then imaging the subject. Examples of labels useful for diagnostic imaging in accordance with the present disclosure are radiolabels such as I¹²³, I¹³¹, I¹¹¹, Tc^{99m}, P³², I¹²⁵, H³, C¹⁴, and Rh¹⁸⁸, fluorescent labels such as fluorescein and rhodamine, nuclear magnetic resonance active labels, positron emitting isotopes detectable by a positron emission tomography (“PET”) scanner, chemiluminescence such as luciferin, and enzymatic markers such as peroxidase or phosphatase. Short-range radiation emitters, such as isotopes detectable by short-range detector probes, such as a transrectal probe, can also be employed. The antibody can be labeled with such reagents using techniques known in the art. For example, see Wensel and Meares, Radioimmunoimaging and Radioimmunotherapy, Elsevier, N.Y. (1983), which is hereby incorporated by reference, for techniques relating to the radiolabeling of antibodies. See also D. Colcher et al., “Use of Monoclonal Antibodies as Radiopharmaceuticals for the Localization of Human Carcinoma Xenografts in Athymic Mice,” Meth. Enzymol. 121: 802-816 (1986), which is hereby incorporated by reference.

Labeled antibodies in accordance with this disclosure can be used for in vitro diagnostic tests to detect cancer antigens shed into the bloodstream (see, e.g., Examples 5-8, described above). The specific activity of an antibody, binding portion thereof, probe, or ligand, depends upon the half-life, the isotopic purity of the radioactive label, and how the label is incorporated into the biological agent. In immunoassay tests, the higher the specific activity, in general, the better the sensitivity. Procedures for labeling antibodies with the radioactive isotopes are generally known in the art.

The radiolabeled antibody can be administered to a patient where it is localized to cancer cells bearing the antigen with which the antibody reacts, and is detected or “imaged” in vivo using known techniques such as radionuclear scanning using e.g., a gamma camera or emission tomography. See, e.g., A. R. Bradwell et al., “Developments in Antibody Imaging,” Monoclonal Antibodies for Cancer Detection and Therapy, R. W. Baldwin et al. (eds.), pp. 65-85 (Academic Press 1985), which is hereby incorporated by reference. Alternatively, a positron emission transaxial tomography scanner, such as designated Pet VI located at Brookhaven National Laboratory, can be used where the radiolabel emits positrons (e.g., C¹¹, F¹⁸, O¹⁵, and N¹³).

Fluorophore and chromophore labeled biological agents can be prepared from standard moieties known in the art. Since antibodies and other proteins absorb light having wavelengths up to about 310 nm, the fluorescent moieties should be selected to have substantial absorption at wavelengths above 310 nm, for example, above 400 nm. A variety of suitable fluorescence and chromophores are described by Stryer. Science, 162:526 (1968) and Brand, L. et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

### Therapy

In one embodiment in accordance with the present disclosure, methods are provided for treatment, monitoring the progress, and/or effectiveness of a therapeutic treatment.

In one embodiment of each of the therapeutic methods described herein, the subject is first diagnosed with cancer. In one embodiment, the subject is first diagnosed with an aggressive cancer. In another embodiment, the subject has a disease chosen from liquid cancers (e.g., MM, ALL, and AML) and solid cancers (e.g., prostate cancer). In one embodiment, the cancer patient is diagnosed as having relapsed. In one embodiment, antibodies herein are used for diagnosing and/or treating the cancer patient. In one embodiment, one or more glycomimetic compounds are used for treating the cancer patient. In one embodiment, the patient is diagnosed as having an aggressive cancer using the antibodies disclosed herein. In one embodiment, the patient is treated with one or more glycomimetic compounds after being diagnosed as having an aggressive cancer using the antibodies disclosed herein.

Certain methods disclosed herein are applicable to any situations wherein identification of E-selectin ligands is desired, for example, in clinical research or for drug discovery.

In one embodiment, described herein is a pharmaceutical composition comprising one or more glycomimetic compounds, and a pharmaceutically-acceptable carrier. In one embodiment, the pharmaceutical composition comprises GMI-1271, and a pharmaceutically-acceptable carrier. In one embodiment, the pharmaceutical composition comprises GMI-1359, and a pharmaceutically-acceptable carrier.

Depending on the specific embodiment, pharmaceutical compositions described herein can include, for example, agents that interfere with the function of the cell surface carbohydrate of aggressive cancer cells so that the cells are unable to bind E-selectin.

Routes of administration for pharmaceutical compositions include, but are not limited to, intradermal, intramuscular, intraperitoneal, intravenous, and subcutaneous routes.

In another embodiment, the subject being treated receives chemotherapy as an adjunct (either before, concurrent with, or after) to administration of the pharmaceutical composition according to the disclosed embodiments. In one embodiment, the subject being treated receives chemotherapy as an adjunct (either before, concurrent with, or after) to administration of a pharmaceutical composition comprising GMI-1271. In one embodiment, the subject being treated receives chemotherapy as an adjunct (either before, concurrent with, or after) to administration of a pharmaceutical composition comprising GMI-1359. In one embodiment, the adjunct chemotherapy treatment comprises administration of bortezomib.

Other embodiments of the disclosure will be apparent to those skilled in the art from consideration of the specification and practice of the disclosure disclosed herein.

What is claimed is:

1. A method of treating a patient with cancer comprising:
obtaining a cancer cell, blood, or blood fraction sample from the patient;
determining whether an antibody with sialyl Le^{a} and sialyl Le^{x} binding domains binds to the cancer cells; and
administering to the patient in need thereof an effective amount of at least one glycomimetic compound if the antibody binds to the cancer cells.

2. The method of claim 1, further comprising administering to the patient chemotherapy and/or radiation therapy.

3. The method of claim 1, further comprising administering to the patient bortezomib.

4. The method of any preceding claim, wherein the at least one glycomimetic compound is chosen from glycomimetics of Formula (I): 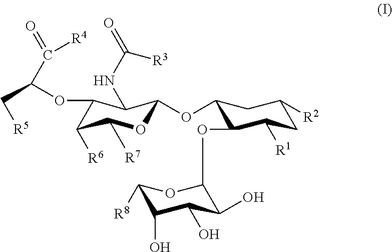
prodrugs of Formula (I), and pharmaceutically acceptable salts of any of the foregoing, wherein
R¹ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R² is chosen from H, a non-glycomimetic moiety, and a linker-non-glycomimetic moiety, wherein the non-glycomimetic moiety is chosen from polyethylene glycol, N-linked cyclam, thiazolyl, chromenyl,
—C(═O)NH(CH₂)₁₋₄NH₂, C₁-C₈ alkyl, and —C(═O)OY groups, wherein Y is chosen from C₁-C₄ alkyl, C₂-C₄ alkenyl, and C₂-C₄ alkynyl groups;
R³ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R⁴ is chosen from —OH and —NZ¹Z² groups, wherein Z¹ and Z², which may be identical or different, are each independently chosen from H, C₁-C₈ alkyl, C₂-C₈ alkenyl, C₂-C₈ alkynyl, C₁-C₆ haloalkyl, C₂-C₈ haloalkenyl, and C₂-C₈ haloalkynyl groups, wherein Z¹ and Z² may join together to form a ring;
R⁵ is chosen from C₃-C₈ cycloalkyl groups;
R⁸ is chosen from —OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R⁷ is chosen from —CH₂OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups; and
R⁸ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups.

5. The method of any preceding claim, wherein the cancer is a leukemia, lymphoma, or a myeloma.

6. The method of any preceding claim, wherein the cancer is characterized by solid tumors.

7. The method of any preceding claim, wherein the antibody is HECA-452.

8. The method of any preceding claim, wherein the at least one glycomimetic compound is chosen from heterobifunctional compounds that are antagonists of E-selectin and CXCR4.

9. The method of any one of claims 1-7, wherein the at least one glycomimetic compound is GMI-1271 or GMI-1359.

10. A method for producing an antibody for identifying cancer stem cells comprising administering cancer cells to a host and screening the resultant population of antibodies for an antibody that binds to sialyl Le^{a} and sialyl Le^{x}.

11. A method of detecting cancer cells expressing sialyl Le^{a} and sialyl Le^{x} comprising:
obtaining from a patient a cancer cell sample; and
detecting whether sialyl Le^{a} and sialyl Le^{x} are present in the sample by contacting the sample with an antibody that binds sialyl Le^{a} and sialyl Le^{x} and detecting binding between sialyl Le^{a} and sialyl Le^{x} and the antibody.

12. A method of diagnosing a patient with cancer comprising:
obtaining from the patient a cancer cell, blood, or blood fraction sample;
detecting whether HECA-452 glycoforms of CD62L are present in the sample by contacting the sample with a HECA-452 antibody and CD62L antibody and detecting binding between the HECA-452 glycoforms of CD62L, the HECA-452 antibody, and the CD62L antibody; and
diagnosing the patient with aggressive cancer when the presence of HECA-452 glycoforms of CD62L in the sample is detected.

13. A method of diagnosing a patient with cancer comprising:
obtaining a cancer cell, blood, or blood fraction sample from the patient;
detecting whether sialyl Le^{a} and sialyl Le^{x} are present in the sample by contacting the sample with an antibody that binds sialyl Le^{a} and sialyl Le^{x} and detecting binding between sialyl Le^{a} and sialyl Le^{x} and the antibody; and
diagnosing the patient with aggressive cancer when the presence of sialyl Le^{a} and sialyl Le^{x} in the sample is detected.

14. A method of diagnosing and treating a patient with cancer comprising diagnosing the patient according to the method of claim 12 or 13 and administering to the patient in need thereof an effective amount of at least one glycomimetic compound.

15. The method of claim 14, further comprising administering to the patient chemotherapy and/or radiation therapy.

16. The method of claim 14, further comprising administering to the patient bortezomib.

17. The method of claim 16, wherein the at least one glycomimetic compound is chosen from glycomimetics of Formula (I): 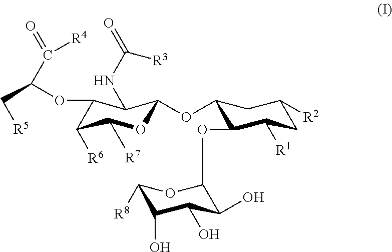
prodrugs of Formula (I), and pharmaceutically acceptable salts of any of the foregoing, wherein
R¹ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R² is chosen from H, a non-glycomimetic moiety, and a linker-non-glycomimetic moiety, wherein the non-glycomimetic moiety is chosen from polyethylene glycol, N-linked cyclam, thiazolyl, chromenyl, —C(═O)NH(CH₂)₁₋₄NH₂, C₁-C₈ alkyl, and —C(═O)OY groups, wherein Y is chosen from C₁-C₄ alkyl, C₂-C₄ alkenyl, and C₂-C₄ alkynyl groups;
R³ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R⁴ is chosen from —OH and —NZ¹Z² groups, wherein Z¹ and Z², which may be identical or different, are each independently chosen from H, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups, wherein Z¹ and Z² may join together to form a ring;
R⁵ is chosen from C₃-C₈ cycloalkyl groups;
R⁶ is chosen from —OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R⁷ is chosen from —CH₂OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups; and
R⁸ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups.

18. The method of any one of claims 14-17, wherein the at least one glycomimetic compound is chosen from heterobifunctional compounds that are antagonists of E-selectin and CXCR4.

19. The method of any of one claims 14-17, wherein the at least one glycomimetic compound is GMI-1271 or GMI-1359.

20. The method of any of one claims 10-19, wherein the cancer is a leukemia, lymphoma, or a myeloma.

21. The method of any one of claims 10-20, wherein the cancer is characterized by solid tumors.

22. The method of any one of claims 11 and 13-21, wherein the antibody is HECA-452.
